SEQUENCE LISTING (18 sequences)

SEQ ID NO: 1 - DNA, 20 bp, artificial
  Primer 1 (beta-hCG gesamt)
tcacttcaccgtggtctccg

SEQ ID NO: 2 - DNA, 20 bp, artificial
  Primer 2 (beta-hCG total)
tgcagcacgcgggtcatggt

SEQ ID NO: 3 - DNA, 23 bp, artificial
  Primer 3 (beta-hCG beta-7, beta-6, beta-6e)
cactgaggggagaggactggggt

SEQ ID NO: 4 - DNA, 23 bp, artificial
  Primer 4 (beta-hCG beta-5, beta-8, beta-3)
cagtgagaggagagggctggggc

SEQ ID NO: 5 - DNA, 861 bp, human
agcactttcctcgggtcacggcctcctcctggttcccaagaccccaccataggcagaggcaggccttcctacaccctactctctgtgcctccagcctcgactagtccctagcactcgacgactgagtctcagaggtcacttcaccgtggtctccgcctcatccttggcgctagaccactgaggggagaggactggggtgctccgctgagccactcctgtgcctccctggccttgtctacttctcgccccccgaagggttagtgtccagctcactccagcatcctacaacctcctggtggccttgacgcccccacaaacccgaggtataaagccaggtacaccaggcaggggacgcaccaaggatggagatgttccaggggctgctgctgttgctgctgctgagcatgggcgggacatgggcatccaaggagatgcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 6 - DNA, 861 bp, human
agcactttcctcgggtcacggcctcctcctggttcccaagaccccaccataggcagaggcaggccttcctacaccctactctctgtgcctccagcctcgactagtccctaacactcgacgactgagtctcagaggtcacttcaccgtggtctccgcctcatccttggcgctagaccactgaggggagaggactggggtgctccgctgagccactcctgtgcctccctggccttgtctacttctcgccccccgaagggttagtgtcgagctcactccagcatcctacaacctcctggtggccttgccgcccccacaaccccgaggtatgaagccaggtacaccaggcaggggacgcaccaaggatggagatgttccaggggctgctgctgttgctgctgctgagcatgggcgggacatgggcatccaaggagccacttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 7 - DNA, 861 bp, human
agcactttyctcgggtcacggcctcctcctggttcccaagaccccaccataggcagaggcaggccttcctacaccctactctctgtgcctccagcctcgactagtccctarcactcgacgactgagtctcagaggtcacttcaccgtggtctccgcctcatccttggygctagaccactgaggggagaggactggggtgctccgctgagccactcctgtgcctccctggccttgtctacttctcgccccccgaagggttagtgtcsagctcactccagcatcctacaacctcctggtggccttgmcgcccccacaamcccgaggtatraagccaggtacaccaggcaggggacgcaccaaggatggagatgttccaggggctgctgctgttgctgctgctgagcatgggcgggacatgggcatccarggagmyrcttcggccacggtgccgccccatcaatgccaccctggctgtggagaaggagggctgccccgtgtgcatcaccgtcaacaccaccatctgtgccggctactgccccaccatgacccgcgtgctgcagggggtcctgccggccctgcctcaggtggtgtgcaactaccgcgatgtgcgcttcgagtccatccggctccctggctgcccgcgcggcgtgaaccccgtggtctcctacgccgtggctctcagctgtcaatgtgcactctgccgccgcagcaccactgactgcgggggtcccaaggaccaccccttgacctgtgatgacccccgcttccaggcctcctcttcctcaaaggcccctccccccagccttccaagtccatcccgactcccggggccctcggacaccccgatcctcccacaataaa

SEQ ID NO: 8 - DNA, 20 bp, artificial
  Primer 8 (beta-hCG beta-5, beta-8, beta-3)
catgggcatccaaggagccg

SEQ ID NO: 9 - DNA, 20 bp, artificial
  Primer 9 (beta-hCG beta-6)
catgggcatccaaggagcca

SEQ ID NO: 10 - DNA, 20 bp, artificial
  Primer 10 (beta-hCG beta-7, beta-6e)
catgggcatccagggagatg

SEQ ID NO: 11 - DNA, 17 bp, artificial
  Primer 11 (total beta-hCG)
tcggggtgtccgagggc

SEQ ID NO: 12 - DNA, 20 bp, artificial
  Primer 12 (beta-hCG beta-5, beta-8, beta-3)
gatgacccccgcttccagga

SEQ ID NO: 13 - DNA, 20 bp, artificial
  Primer 13 (beta-hCG beta-7, beta-6)
gatgaccccccgttccaggc

SEQ ID NO: 14 - DNA, 17 bp, artificial
  Primer 14 (total beta-hCG)
tcgggtcacggcctcct

SEQ ID NO: 15 - DNA, 22 bp, artificial
  Primer 15 (beta-hCG beta-5, beta-8, beta-3)
acggcctcctcctggctcccag

SEQ ID NO: 16 - DNA, 22 bp, artificial
  Primer 16 (beta-hCG beta-7, beta-6, beta-6e)
acggcctcctcctggttcccaa

SEQ ID NO: 17 - protein, 165 aa, human
MEMFQGLLLLLLLSMGGTWASKEMLRPRCRPINATLAVEKEGCPVCITVNTTICAGYCPTMMRVGVLQLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

SEQ ID NO: 18 - protein, 165 aa, human
MEMFQGLLLLLLLSMGGTWASREMLRPRCRPINATLAVEKEGCPVCITVNTTICAGYCPTMMRVGVLQLPALPQVVCNYRDVRFESIRLPGCPRGVNPVVSYAVALSCQCALCRRSTTDCGGPKDHPLTCDDPRFQASSSSKAPPPSLPSPSRLPGPSDTPILPQ

(19) United States
(12) Patent Application Publication
(10) Pub. No.: US 2006/0292567 A1
(43) Pub. Date: Dec. 28, 2006

(54) Method and means for determining specific conditions or changes in the uterine epithelium and in the epithelium of other organs

(75) Inventors: Gerolf Zimmermann, Leipzig (DE); Henry Alexander, Leipzig (DE)

Correspondence Address: GUDRUN E. HUCKETT DRAUDT, LONSSTR. 53, WUPPERTAL, 42289, DE

(73) Assignee: Universitat Leipzig, Leipzig (DE)

(21) Appl. No.: 10/540,215

(22) PCT Filed: Dec. 19, 2003
(86) PCT No.: PCT/DE03/04293

§ 371 Date: Jun. 20, 2005

(30) Foreign Application Priority Data
Dec. 21, 2002 (DE) 102 60 556.4
Jun. 6, 2003 (DE) 103 25 637.7
Jun. 6, 2003 (DE) 103 25 636.9

(51) Int. Cl. C12Q 1/68; C07H 21/04

(52) U.S. Cl. 435/6; 536/24.3

(57) ABSTRACT

In a method for determining specific conditions or changes in the endometrium or in the epithelium of other organs, RNA from a blood sample or tissue sample isolated and the expression or over expression of mRNA of at least one of β7-hCG, β6-hCG, and β6e-hCG is measured quantitatively in the blood or tissue sample. Additionally, total βhCG mRNA expression or mRNA expression of at least one of β5-hCG, β8-hCG, β3-hCG is quantitatively measured and brought into relation with the expression or over expression of mRNA of at least one of β7-hCG, β6-hCG, and β6e-hCG.

The invention concerns a method and means for determining specific conditions or changes in the uterus. Conditions of the uterine epithelium or epithelium of other organs that are to be determined in particular by the invention are the receptivity of the endometrium for the implantation of an embryo or neoblastic and tumorous changes. The field of application is medicine, particularly gynecology and oncology.

Human chorionic gonadotropin (hCG) is a hormone whose concentration is increased during pregnancy and which is tested for in pregnancy tests. hCG is comprised of two subunits α-hCG and β-hCG bonded non-covalently. One gene is known for the subunit α-hCG (chromosome 6q21.1-q 23). For the subunit β-hCG seven genes β8, β7, β6, β5, β3, β1 and β2 are known (chromosome 19q13.3).

During pregnancy by means of trophoblasts of the uterus larger amounts of hCG dimer and free α-hCG and β-hCG molecules are formed and secreted into the blood. However, in some non-trophoblastic tissues hCG or its subunits are expressed in minimal quantities (2-6). In the blood of healthy humans who are not pregnant hCG concentrations of hCG up to 1,000 pg/ml and of β-hCG of up to 100 pg/ml are therefore observed (7,8). Higher β-hCG serum values indicate a gonadal or non-gonadal tumor and characterize an unfavorable prognosis as described in connection with lung, bladder, prostate, colon, kidney cell or mamma carcinoma (5, 9-13).

Embryonic trophoblastic tissue expresses almost exclusively hCG β5, β8 and β3. These β-hCG subunits are therefore also referred to as trophoblastic β-hCG (tβ-hCG) or type I-β-hCG.

hCG β7 and β6 are expressed only minimally in some non-trophoblastic tissues, e.g., mamma, lung, prostate, skeletal muscles, bladder, colon, uterus (17). These β-hCG subunits are therefore also referred to as non-trophoblastic β-hCG or type

While the subunits of the type II-β-hCG (β5, β8, and β3) contain aspartate (Asp, D) at position 117 (exon 3) of the amino acid sequence, the type I-β-hCG (β7 and β6) contains alanine (Ala, A) at position 117.

In the past several, studies have been performed with the goal of detecting the β-hCG transcripts in different normal and neoplastic tissues of non-trophoblastic origin by means of a semi-quantitative method (5, 11, 12, 18). These methods show that β-hCG is transcribed in the normal placenta (19), healthy testes (6), but also neoplastic testes (20) and neoplastic bladder tissue (21). In these studies, however, no differentiation is made between the type I-β-hCG and the type II-β-hCG.

In one work (9) the presence of hCG β7 in healthy and of hCG β8, β5, β3 in malignant bladder tissue is detected by specific restriction enzymes for detecting individual transcripts.

In a further work, the over expression of the type II-β-hCG (β5, β8, β3) in malignant transformed non-trophoblastic tissue is determined by means of the determined transformation index that is defined by the ratio between the gene expression of hCG β5, β8, β3 to the total expression of all β-hCG in the same tissue. The index is determined by means of primers between exon 2 and exon 3 that detect the point mutation C117 in the C-terminal region of the β-hCG in exon 3 (17). In the past, this point mutation Asp-Ala at position 117 of the β-hCG amino acid chain is used in the afore mentioned quotient as a diagnostic parameter of the neoplastic transformations.

A tumor identification by analysis of the secretion products, in particular by utilizing the type II-β-hCG as indicator for cancer, was demonstrated already in 1996 by a French research group. Bellet et al. (17) describe that the β subunit of hCG is coded by four non-allelic β-hCG genes. The important findings include that the malignant transformation of non-trophoblastic tissues is always connected with the expression of the β-hCG to the gene that are usually transcribed in the trophoblast. The research of the β-hCG genes that are expressed by non-trophoblastic tissue leads to the result: normal non-trophoblastic tissue expresses mainly β-hCG gene of the type I (hCG β7, β6) while upon malignant transformation β-hCG genes of the type II (hCG β5, β8, β3) are expressed also.

In U.S. Pat. No. 6,194,154, a method for determining the malignant transformation of human cells is disclosed that compares the over expression of hCG β3, β5, β8, and β9-mRNA in malignant cells with the expression of hCG β7, β6 in non-malignant cells. An increase of the mRNA expression of hCG β3, β5, β8, and β9 in relation to the total β gene expression in the malignant cells is also determined. Moreover, it is disclosed that the point mutation in the mRNA nucleotide sequence of position 775 indicates C for β5, β8, β3 is A and for β7, β6 and therefore codes aspartate (Asp, A) or alanine (Ala, and A) in the amino acid position 117. Based on this, a test kid is provided that is widely used.

WO 0190344 makes reference to the promoter, enhancer, and other regulators that control the expression of the protein β-hCG in testicular carcinoma. Moreover, discussions regarding gene therapy by introducing promoter gene β-hCG DNA into different cells, for example, in liposomes. The β-hCG protein is used in different tumor tissues as a diagnostic parameter.

It is an object of the invention to provide a method and means for determining specific conditions or changes in the uterus and in other organs, in particular, the endometrium but also in the epithelia of other organs. Conditions of the uterus to be determined in particular by means of the invention are the receptivity of the endometrium for the implantation of an embryo or neoplastic or tumorous changes.

According to the invention, the object is solved by a method for determining specific conditions or changes in the uterus in which method mRNA is isolated from a blood sample and/or tissue sample and in this sample a quantitative measurement of the mRNA gene expression of β7-hCG and/or β6-hCG and/or β6e-hCG.

β6-hCG has the gene sequence (cDNA) according to SEQ ID NO. 5 and β7-hCG according to SEQ ID No. 6.

β6e-hCG is a variant of the type I-β-hCG (β6 or β7) that, surprisingly, has been newly detected and has a gene sequence (cDNA) according to SEQ ID NO. 7. The β6e-hCG gene is expressed in the endometrium and codes for a protein according to SEQ ID NO. 17 or SEQ ID NO. 18.

In a preferred variant of the method according to the invention as an internal standard the total βhCG-mRNA gene expression or the mRNA gene expression of individual or all type II-β-hCG subunits (β5-hCG, β8-hCG, β3-hCG) is measured. The mRNA gene expression of β7-hCG and/or β6-hCG and/or β6e-hCG is brought into relation with the reference standard for evaluation purposes.

Preferably, the quantitative measurement of the mRNA gene expression is carried out by means of quantitative RT-PCR. In the known process of RT-PCR, first by means of the enzyme reverse transcriptase (RT) the complementary DNA (cDNA) is synthesized based on the isolated RNA. As a primer for the RT an oligonucleotide having a poly-dt sequence is selected (oligo-dT). The oligo-dT is composed preferably of 10 to 20 deoxythymidine (dT) monomers. Individual cDNAs are amplified in the subsequent PCR with a sequence-specific primer pair.

In this connection, the sequence of at least one primer is selected preferably such that the primer hybridizes with a β-hCG-cDNA sequence that is formed by the combination of two exons. By this selection it is achieved that by means of the primer only cDNA is amplified but not contaminants of genomic β-hCG-DNA that are possibly present in the sample.

As an external standard, preferably a defined amount of mRNA or even cDNA of β7-hCG or β5-hCG is used in a parallel measurement carried out under identical conditions.

It is especially preferred to perform the PCR as a real-time PCR. Known real-time PCR methods are, for example, TaqMan, FRET (fluorescence resonance energy transfer) and Beacon methods. By employing fluorescence-marked primers, in this method the PCR product can be quantified advantageously during the PCR.

The invention claims also the real-time measurement as one tube RT-PCR or the use of other methods for quantitative detection of the expression of specific gene copies in addition to SYBR Green I, for example, the use of gene-specific oligonucleotides as hybridization samples with different dye or fluorescence marker binding (TaqMan, FRET, Beacon).

In an especially preferred variant of the method according to the invention, based on the cDNA obtained by reverse transcriptase (RT) the total βhCG-cDNA is amplified in a first PCR step with at least one first primer pair.

The amplification of the total βhCG is achieved in that this first primer pair hybridizes with the cDNA of the type II-β-hCG subunits (β5-hCG, β8-hCG, β3-hCG) as well as of the type I-β-hCG subunits (β7 and β6 and β6e).

In a subsequent second PCR step, the cDNA of individual or all type I-β-hCG subunits (β7, β6, β6e) is specifically amplified with at least one third primer.

In order to amplify in the second step only type I-β-hCG subunits and not type type II-β-hCG subunits, the third primer is selected such that it specifically hybridizes only with cDNA of β7-hCG and β6-hCG and β6e-hCG but not with cDNA of β5-hCG, β8-hCG, and β3-hCG.

Preferably, in the second PCR step, i.e., a so-called nested PCR, the cDNA of at least one or several type II-β-hCG subunits ((β5-hCG and/or β8-hCG and/or β3-hCG) is specifically amplified additionally with at least one fourth primer. For this purpose, the fourth primer is selected such that it specifically hybridizes with the cDNA of β5-hCG, β8-hCG, β3-hCG but not with the cDNA of β7-hCG and β6-hCG and β6e-hCG.

The primer for the second step of PCR can be added before or after performing the first step of the PCR.

The third and fourth primers are preferably provided with different marker molecules that enable differentiation between the PCR products that are formed by amplification with the third and fourth primer.

Preferably, as a first primer of the first primer pair for amplification of the total βhCG a DNA oligonucleotide of exon 1 of the βhCG is selected that has a length of 10 to 30 base pairs. Such a preferred primer has the sequence according to SEQ ID NO. 1.

Preferably, as a second primer of the first primer pair for amplification of the total βhCG a DNA oligonucleotide of the complementary sequence of exon 3 of the βhCG is selected that has a length of 10 to 30 base pairs. Such a preferred primer has the sequence according to SEQ ID NO. 2.

Further preferred primers for the primer pair for amplification of total βhCG are primers having the sequences according to SEQ ID NO. 11 and SEQ ID NO. 14.

As a third primer for specific amplification of type I-βhCG, preferably a DNA oligonucleotide of the area of the β7-hCG is selected that has a length of 10 to 30 base pairs. A preferred primer has the DNA sequence according to SEQ ID NO. 3. Further preferred primers for specific amplification of the type I-βhCG are primers with sequences according to SEQ ID NO. 9, SEQ ID NO. 10, SEQ ID NO. 13, and SEQ ID NO. 16.

As a fourth primer for specific amplification of the type II-βhCG preferably a DNA oligonucleotide of the area of the β5-hCG is selected that has a length of 10 to 30 base pairs. Such a preferred primer has a DNA sequence according to SEQ ID NO. 4. Further preferred primers for specific amplification of the type II-βhCG are primers having sequences according to SEQ ID NO. 8, SEQ ID NO. 12, and SEQ ID NO. 15.

For the preferred real-time PCR at least one of the primers is fluorescence-marked. It is especially preferred that the third primer is provided with such a fluorescence marker in order to enable quantification of the amplified type l-βhCG cDNA during PCR.

Preferably, one of the two primers of the first primer pair and optionally the fourth primer are provided with fluorescence markers wherein, however, the markers of these primers relative to one another and to the primer 1 differ with regard to their adsorption and/or emission spectrum.

With these different fluorescence markers a parallel quantification of the amplified type I-βhCG cDNA and optionally type II-βhCG cDNA during PCR and a comparison with the total βhCG cDNA is possible.

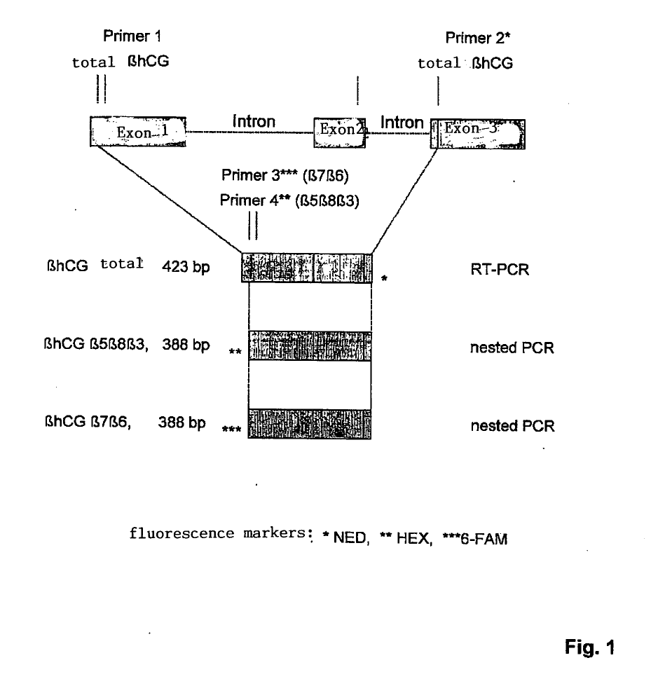

The method according to the invention is explained with the aid of the flowchart of FIG. 1. The primer 1 employed in FIG. 1 (amplification of total βhCG) is not marked, primer 2 (amplification of total βhCG) contains the fluorescence marker NED.

The primer 3 employed in FIG. 1 for the amplification of type I-β-hCG (β7, β6, β6e) is marked with 6-FAM. Primer 4 for the amplification of the type II-β-hCG (β5, β8, β3) contains the fluorescence marker HEX.

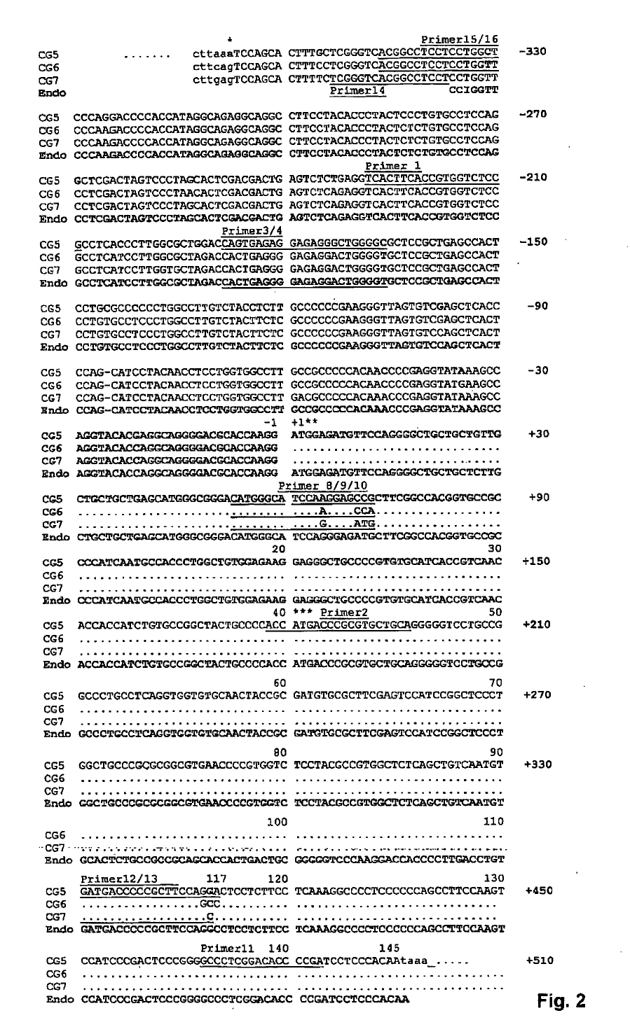

The selection of the primers is illustrated in FIG. 2.

FIG. 2 shows a sequence alignment of the sequences β5-hCG (“CG5”), β7-hCG (“CG6”), β7-hCG (“CG7”) and β7-hCG (“endo”). The * indicates the start of transcription, ** indicates the start of translation.

The numerals above the nucleic acid sequences indicate the amino acid positions of the coded proteins. Underscoring indicates the sequence areas which are hybridized with the primers.

The oligonucleotide primer pairs 1 and 2, according to SEQ ID NO. 1 and NO. 2, 1 and 11, according to SEQ ID NO. 1 and NO. 11, as well as 14 and 2, according to SEQ ID NO. 14 and NO. 2 of the sequence listing have been selected such that, by employing the total RNA and the RT-PCR method, the sum of all βhCG transcripts β5, β8, β3 and also β7, β6 are represented with the same efficiency in a first amplification step. These mentioned primer pairs exclude the βLH amplification because of different nucleotide sequences.

In the subsequent nested PCR step or by employing the real-time RT-PCR quantification method, the transcript β7, β6, β6e is amplified by using the primers 3 and 2, primers 9 or 10 and 2, primers 13 and 11, as well as primers 16 and 12, and the transcript β5, β8, β3 is amplified with primers 4 and 2, primers 8 and 2, primers 12 and 11, as well as primers 15 and 2.

By means of the primer 9 having the sequence according to SEQ ID NO. 9, only β6-hCG is amplified but not β7-hCG and β6e-hCG.

By means of the primer 10 having the sequence according to SEQ ID NO. 10, only β7-hCG and β6e-hCG are amplified but not β6-hCG.

By means of the primer 13 having the sequence according to SEQ ID NO. 13, only β7-hCG and β6-hCG are amplified but not β6e-hCG.

By a parallel amplification of cDNA with the primers 9, 10, and 13 (having the sequences SEQ ID NO. 9 and/or SEQ ID NO. 10 and/or SEQ ID NO. 13) it is surprisingly possible to differentiate between the mRNA expression of β7-hCG and β6-hCG and β6e-hCG.

For this purpose, preferably a RT-PCR is performed that corresponds to the above described method for differentiating the expression of type I and type II β-hCG with the difference that in the second step two differently marked primers of the group of sequences SEQ ID NO. 9, SEQ ID NO. 10, SEQ ID NO. 13 are used.

The invention provides a model for tumor-specific gene transcription especially of a new promoter βhCG that becomes active only in different tumor tissues including but not limited to testicular carcinoma. The invention provides also methods for analyzing the promoter expression type I-β-hCG subunits and type II-β-hCG subunits. For this purpose, PCR primers 15 and 16 (SEQ ID NO. 15, SEQ ID NO. 16) that hybridize with the promoter area is carried out.

For performing the method according to the invention by means of RT-PCR, a diagnostic kit is preferably used containing an amount of the following components, respectively:

1. For the cDNA synthesis:
a) oligo-dT
b) reverse transcriptase

2. For the PCR:
c) at least two primers that hybridize with cDNA of one or several type II-β-hCG subunits type I-β-hCG (β7, β6, β6) wherein at least one of the two primers is sequence-specific for type I-β-hCG, i.e. it does not hybridize with type II-β-hCG (β5, β8, β3);
d) a DNA polymerase resistant above 80° C., e.g. taq polymerase;
   as well as appropriate reaction buffers.

Compositions of such reaction buffers are known to a person skilled in the art and contain customarily RNase inhibitors and as building blocks for the polymerase dNTPs, as well as a quantity of bivalent cations, for example, Mg²⁺.

Preferably, the diagnostic kit comprises an amount of a first primer pair that hybridizes with cDNA of the type II-β-hCG (β5, β8, β3) as well as type I-β-hCG (β7, β6, and β6e) and a third primer that is sequence-specific for type I-β-hCG, i.e., does not hybridize with the type II-β-hCG (β5, β8, β3).

Preferably, as a first primer of the first primer pair for amplification of the total βhCG a DNA oligonucleotide of exon 1 of the βhCG is selected that has a length of 10 to 30 base pairs. A thus preferred primer has the DNA sequence according to SEQ ID NO. 1.

Preferably, as a second primer of the first primer pair for amplification of total βhCG a DNA oligonucleotide of the complementary sequence of exon 3 of the βhCG is selected that has a length of 10 to 30 base pairs. Such a preferred primer has the DNA sequence according to SEQ ID NO. 2.

Further preferred primers for the primer pair for amplification of total βhCG are primers with sequences according to SEQ ID NO. 11 and SEQ ID NO. 14.

As a third primer for specific amplification of the type I-βhCG, preferably a DNA oligonucleotide of the (please supplement) area of the β7-hCG is selected that has a length of 10 to 30 base pairs. Such a preferred primer has the DNA sequence according to SEQ ID NO. 3.

In a preferred embodiment, the diagnostic kit contains an amount of a fourth primer that hybridizes specifically with the cDNA of the type I-β-hCG but not with the cDNA of the type II-β-hCG. Further preferred primers for specific amplification of the type I-βhCG are primers having sequences according to SEQ ID NO. 9, SEQ ID NO. 10, SEQ ID NO. 13, and SEQ ID NO. 16.

As a fourth primer for specific amplification of the type II-βhCG preferably a DNA oligonucleotide of the (please supplement) area of the β5-hCG is selected that has a length of 10 to 30 base pairs. Such a preferred primer has the DNA sequence according to SEQ ID NO. 4. Further preferred primers for specific amplification of the type II-βhCG are primers having sequences according to SEQ ID NO. 8, SEQ ID NO. 12, and SEQ ID NO. 15.

Preferably, at least one of the primers is fluorescence-marked. This enables performing real-time PCR.

Especially preferred, a primer of the first primer pair, the third primer, and optionally the fourth primer are to be provided with fluorescence markers that differ from one another with regard to their adsorption and/or emission spectra.

The primer sequences having the sequences according to SEQ ID NO. 3 and SEQ ID NO. 4 as well as SEQ ID NO. 8 to SEQ ID NO. 16 are also an aspect of the invention.

The method is employed in accordance with the invention for determining specific conditions or changes in the uterus. With the method according to the invention, the receptivity of the endometrium for the implantation of an embryo or instead neoplastic and tumorous changes can be determined.

A preferred use of the method is the use for diagnosing the receptivity of the endometrium (implantation diagnostic).

The term diagnostic of the receptivity of the endometrium is to be understood in the context of the present invention as the determination of optimal implantation conditions, i.e., recognizing the possibility that for a fertilized egg in the endometrium optimal conditions are present for embedding and for growing there subsequently.

The invention is based on the scientific finding that the level of expression of the genes of the type I-β-hCG (β7, β6, eβ6) in the normal secretory epithelium of the uterus lining (endometrium) or in the mononuclear cells of the peripheral blood represents a reliable indicator for a possible successful implantation. The higher the expression the better the chances for a successful implantation of a fertilized egg or an embryo.

The use in accordance with the present invention is based on the scientific finding that a reliable indicator for a possible successful implantation is the evaluation of the proportion of the expressed 5′-non-translating promoter sequences of the βhCG (exon 1) of βhCG gene β7, β6, as an absolute value or relative to β5, β8 β3.

The gene hCG β7 and β6 of the gene cluster are expressed mainly in the normal secretory epithelium of the endometrium. The genes hCG β5, β8, β3 of the gene cluster are expressed in the normal trophoblast and in the carcinoma-transformed epithelium. Lymphocytes (CD3), natural killer cells, and monocytes (CD14) express hCG β5 in normal persons.

For the inventive diagnostic of receptivity of the endometrium the determination of the expression of hCG and of the allelic gene β7 is required. It has been recognized that the contents of β6 hCG and β7 hCG in the body's own epithelial tissue or blood cells determines the success of an implantation fundamentally and that therefore the knowledge of the amount of hCG β7 and β6, considered absolute or relative in knowledge of the quotient of hCG β7, β6 as numerator and hCG β5, β8, β3 as denominator provides information in regard to the promising moment of implantation.

For determining the proportion of hCG β7, β6, β6e and of hCG β5, β8, β3, the quantitative RT-PCR is suitable.

For diagnosing the receptivity of the endometrium, preferably tissue from the endometrium or from the cervical lining or peripheral blood is removed from the female patient and the analysis of the mRNA expression is determined in this blood or tissue sample with the method according to the invention. Based on the level of the determined mRNA expression of β7-hCG and/or β6-hCG and/or β6-hCG it is then possible to draw conclusions in regard to the receptivity of the uterine for an embryo in the current or the subsequent cycle.

For this purpose, four to six days after ovulation, cells are collected by means of a mini catheter from the uterine cavity, by means of a cotton swab from the cervical channel or by means of a wooden tongue depressor from the oral mucous membrane, or peripheral EDTA blood or heparin blood is removed. From the collected cells the mRNA β-hCG is isolated, cDNA is produced by RT-PCR, and the cDNA is amplified and determined quantitatively.

The production and amplification of the cDNA from mRNA is realized preferably by real-time measurement in a one-tube RT-PCR. Alternatively, other methods are used for the inventive quantitative determination of the expression of specific gene copies, preferably by utilization of gene-specific oligonucleotides as hybridization samples with different dye marker or fluorescence marker bonding (TaqMan, FRET, Beacon).

A positive detection of mRNA of β6-hCG, β7-hCG or eβ6hCG indicates that the endometrium differentiates in the direction toward implantation readiness.

A further preferred use of the method is the use for retrospective diagnostic of receptivity of the endometrium. The term retrospective implantation diagnostic in the context of the present invention is to be understood such that in the past cycle optimal implantation conditions were present. By means of the information on the implantation conditions in the past cycle, prognoses in regard to the implantation conditions, i.e., the receptivity of the uterine for a fertilized egg or an embryo, in the following cycle can be made.

For a retrospective diagnostic of the receptivity of the endometrium in principle the same procedure as for preparatory implantation diagnostic is carried out with the difference that the analysis of β6 and β7 hCG expression is carried out in a sample of menstrual blood. In the menstrual blood, there are sufficient cells of the endometrium enabling an analysis.

The advantage of analysis in the menstrual blood relative to the afore described method resides in that it is not invasive. It is neither necessary to draw peripheral blood nor to take a tissue sample from the uterus.

A further preferred use of the method is the use for tumor diagnostics.

The use in accordance with the invention is based on the scientific finding that a reliable indicator for the presence and the growth of tumor cells is the evaluation of the proportion of expressed 5′-non-translating promoter sequences of the βhCG (exon 1) of βhCG gene β7, β36 to β5, β8, β3 which differ within this gene section with regard to a plurality of nucleotide differences.

In contrast to mutation of a single nucleotide in the codon 117 of the afore described C117 assay (exon 3), the βhCG genes β7, β6 differ from those of β5, β8, β3 in this gene section of the βhCG promoter gene (exon 1) by a high number of nucleotides, between gene 5 and gene 7 by n=20 and with the selected primers n=12. Moreover, with the included exon 1 the possibly falsifying proportion of the gene expression hCG β1 and β2 for the total expression of all βhCG is prevented.

Preferably, for detecting uterus carcinoma, tissue of endometrium or cervix is removed from a female patient and the mRNA expression in this tissue sample is analyzed with the method according to the invention.

Preferably, the values of the mRNA expression in the tumor tissue are compared to the values of the mRNA expression in the healthy tissue.

In an especially preferred variant of the use, the value of the mRNA expression of the β7-hCG and/or β6-hCG and/or β6e hCG is divided for this purpose by the sum of the expression mRNA expression of the total βhCG and, based on the value of the thus obtained quotient, conclusions in regard to the level of malignancy of the tumor are drawn.

It was found that in some neoplastic and tumorous non-trophoblastic tissues hCG β5, β8, β3 are expressed increasingly and in the neoplastic trophoblast additionally hCG β7, β6 are expressed.

The invention will be explained in more detail in the following by means of embodiments without being limited to these embodiments. It is shown in:

Embodiment 1: RT-PCR with fluorescence-marked primers for diagnostic of the receptivity of the endometrium for implantation of an embryo.

Embodiment 2: RT-PCR with non-marked primers for diagnostic of the receptivity of the endometrium for implantation of an embryo.

Embodiment 3: RT-PCR with non-marked primers for retrospective diagnostic of the receptivity of the endometrium for implantation of an embryo.

Embodiment 4: RT-PCR with non-marked primers for for tumor diagnostic.

Embodiment 5: RT-PCR with fluorescent-marked primers for tumor diagnostic.

Embodiment 6: RT-PCR with fluorescence-marked primers for tumor diagnostic.

### EMBODIMENT 1

For diagnostic of the receptivity of the endometrium, cells from the uterine cavity are removed with a mini catheter from the female patient or by means of a cotton swab from the cervix or by means of a wooden tongue depressor from the oral mucous membrane. The cells are immediately frozen and stored at −80° C. until further processing. For analysis, a TRIZOL RNA extraction is performed from the removed cells, the cDNA of the endometrial hCG is amplified specifically in the subsequent RT-PCR process, and quantitatively determined.

It can be assumed that the presence of hCG β7, β6, and β6e is an indicator for an optimal implantation. The lack of hCG β7, β6, and β6e indicates the opposite: the possibility of implantation in this cycle is not to be expected. Especially important is the fact that with the βhCG diagnostic the lack of endometrium or highly built-up secretory endometrium can be detected so that the diagnosis also provides a therapeutic indication. It should be noted that the hCG β6 and β6e can be substantially represented by hCG β7 (six nucleotide differences in exon 1 relative to 24 nucleotides differences between β7 and β5). By means of the selection of different primers provided in the sequence listing, the hCG β7, β6, β6e proportions can be determined in sum but also directly for hCG β7 and β6. On the other hand, the detection of minimal to increased hCG β5, β8, and β3 in the endometrial tissue or its cells can represent an indication of a tumor decease. The tissue samples can also be obtained in analogy to the method of fractioned curettage.

Endometrial tissue (10-30 mg) or cells of this origin are frozen immediately after removal in liquid nitrogen or at −80° C. For examining the three expressed proportions hCG β7, β6, and β6e as well as hCG β8, β5, β3 and total hCG, the total RNA is extracted with TRIZOL and approximately 1 μg of RNA is reverse transcribed for 60 minutes at 42° C. under standard conditions and use of oligo-dT(15) primer.

In this embodiment, the proportion of the gene-specific expressed βhCG amplified material β7, β6, β6e in the endometrium is evaluated relative to the total hCG contents of hCG β7, β6 plus hCG β5, β8, β3 for the diagnostic of the receptivity of the endometrium. For this purpose, the nested RT-PCR method is used which in the first RT-PCR step measures the total proportion of βhCG with specific primers and the fluorescence marker 1 and, in the subsequent nested PCR step with this product, measures hCG β7, β6, β6e with fluorescence marker 2 as well as hCG β5, β8, β3 with fluorescence marker 3. A software program calculates as a quotient the proportion of hCG β7, β6, β6e relative to the total proportion of βhCG.

Use of methods: tissue removal for diagnostics, storage in liquid nitrogen, RNA extraction (23), RT-PCR with fluorescence-marked primer pair, detection of total βhCG expression β5, β8, β3, and β7, β6 and β6e via exon 1, exon 2, and exon 3, nested PCR method with different fluorescence-marked primers for the β7, β6, β6e and possibly β5, β8, β3 proportions, respectively; quantitative evaluation as quotient of β7, β6, β6e fluorescence proportion to total βhCG proportion β7, β6 plus β5, β8, β3 proportion for evaluating the highly built-up secretory endometrial tissue, result 1 for normal tissue and result >0 to 1 for below-value or lacking secretory transformed tissue in embodiment 1; or absolute quantitative evaluation of the expressed copy numbers for the gene-specific βhCG amplified materials β7, β6, β6e and total βhCG according to real-time RT-PCR in comparison to βhCG sequence-specific calibrators for non-fluorescence marked primers and use of standard methods for the evaluation of the normal and neoblastic tissue as in embodiment 2.

Use of devices and material: tissue in liquid nitrogen, Ultra Turrax tissue homogenization, TRIZOL RNA extraction, RT-PCR on thermocycler, fluorescence measurement of the cDNA amplified material on DNA sequencer ABI 373A, software Genescan 672 fragment analysis for evaluation, liquid nitrogen, TRIZOL, cDNA synthesis kit, PCR amplification kit, βhCG primer for total βhCG amplification and nested PCR for β7, β6, β6e and β5, β8, β3, partially fluorescence-marked.

Description of the method for embodiment 1: extraction for total RNA: the fresh tissue material is frozen immediately after removal in liquid nitrogen. The total RNA is extracted by the method of Chomczynski and Sacchi (24), the obtained RNA is quantified spectrophotometrically at 260 nm/280 nm, immediately processed further or stored at −80° C.

Reverse transcription: 1 μg total RNA is transcribed in a reaction mix with the total volume of 5 μl according to standard method: 10 mM Tris-HCl, pH 8.3, 50 mM KCl, 5 mM MgCl₂, 1 mM each of dNTP (dATP, dTTP, dCTP, dGTP), 200 ng oligo-dT primer pdT15, 12.5 U RNase inhibitor, 2.5 U AMV revertase. Incubation of the reaction mixture for 10 minutes at 25° C. (hybridization of the primer), 30 minutes at 42° C. (reverse transcription) and 5 minutes at 95° C. (denaturization of the revertase and of the RNase inhibitor) as well as cooling to 4° C.

PCR amplification of the total βhCG transcripts: In the same tube the PCR mix of 20 μl is added to the cDNA product in the total volume of 25 μl for the amplification of the total βhCG transcript: final concentration of 10 mM Tris-HCl at pH 8.3, 50 mM KCl, 1.5 mM MgCl₂, 200 μM dNTP, 5 pmol each of the two selected primers and 2.5 U Taq DNA polymerase. The amplification conditions, after a prior 3 minute incubation at 95° C., are as follows: 30 seconds at 95° C., 30 seconds at 60° C., 60 seconds at 72° C. for 35 cycles with final 7 minutes at 72° C. and fast cooling to 4° C.

Nested PCR for βhCG β7, β6 and β5, β8, β3 transcripts: 2 μl of the PCR product diluted 1:10,000 are added to a total volume of 20 μl in a PCR mix with a final volume of 10 mM Tris-HCl, pH 8.3, 10 mM KCl, 3 mM MgCl₂, 50 μM dNTP, 0.1 pmol primer 2, 0.1 pmol primer 3, 0.1 pmol primer 4 and 2 U Taq DNA polymerase (Stoffel fragment). The reaction was carried out for five cycles on a thermocycler for 30 seconds at 95° C. and 30 seconds at 65° C., respectively. The nested PCR reaction is performed also with Taq DNA polymerase at standard conditions.

The obtained product contains the two amplification products for βhCG β7, β6, β6e and possibly of hCG β5, β8, β3 with a different fluorescence marker for primer 4 and primer 3, respectively, and both transcripts contain in addition a third common fluorescence marker of the primer 2.

For the analysis on the DNA sequencer (Perkin-Elmer model 373A or a comparable model) 2.5 μl of the product with 2 μl loading buffer and 0.5 μl Genescan size marker and subjected to the electrophoresis at 8% acrylamide, 6M urea, and TBE buffer for one hour. The results are analyzed with the Genescan 672 software (Perkin-Elmer) by employing the obtained fluorescence values for total βhCG transcripts and the β7, β6, β6e and possibly β5, β8, β3 fragments.

The transcription index is calculated in accordance with the method disclosed in Bellet et al. (17).

### EMBODIMENT 2

In this embodiment, for the diagnostic of the receptivity of the endometrium the absolute quantitative evaluation of the expressed copy numbers for the gene-specific βhCG amplified material β7, β6, β6e and possibly βhCG β5, β8, β3 according to real-time RT-PCR in comparison to βhCG-specific calibrators for non-fluorescence-marked βhCG primers is illustrated for the evaluation of the normal highly built-up or below-value or lacking secretorily transformed endometrial tissue.

For the quantitative determination of the three above-mentioned βhCG expression proportions, the real-time PCR on Light Cycler (Roche) or comparable devices of other firms such as Applied Biosystems is used for the amplification of the tumor cDNA. For the synthesis of the RNA standards of the three βhCG expression proportions β7, β6, β6e and possibly β5, β8, β3 and the total βhCG, the three corresponding calibration fragments are amplified under standard PCR conditions from endometrial, placental and tumor-cDNA. For this purpose, again the three aforementioned and now unmarked forward hCG primers (primers 1, 3, 4 or others) that are specific for βhCG type II (β8, β5, β3), βhCG type I (β7, β6), and total βhCG are used with the common reverse βhCG primer (primer 2 or others). The obtained PCR products are cloned in the plasmid vector pGEM-T. By employing the T7 promoters and Sp6 promoters of the pGEMT-T vectors, the plasmid serves as a template for the in-vitro formation of RNA in accordance with the manufacturer's protocol. The formed standard RNAs are cleaned and the concentration is measured.

The real-time PCR amplification on the Light Cycler (Roche) or ABI systems (Applied Biosystems) determines the number of formed gene copies for the two gene-specific βhCG expression groups type II (β8, β5, β3) and type I (β7, β6) as well as total βhCG in the endometrial tissue and in the RNA standards and by employing the primers 8, 9, 10 against 2 the individual proportions of β5, β6 and β7 can be detected also and quantified as an absolute value. The PCR reaction is carried out in the 20 μl reaction volume in the final concentrations of 1× PCR buffer of 50 mM Tris-HCl (pH 8.3), 200 μM dNTPs, with 0.5 μM of the specific forward and reverse βhCG primers, respectively, 4 to 5 mM MgCl₂, 0.5 U Taq polymerase, SYBR Green I with 1:3,000 of the master solution (Molecular Probes) and 1 μl of the templates (endometrium cDNA against standards of known concentration). Other methods of the real-time RT-PCR (TaqMan, FRET, Beacon) can be used alternatively.

### EMBODIMENT 3

For a retrospective diagnostic of the receptivity of the endometrium for implantation of an embryo, menstrual blood is taken from the patient and the corpuscular cell parts are centrifuged off. The cells are immediately frozen and stored at −80° C. until further processing. For analyzing the mRNA expression of the endometrial βhCG the same procedure as in embodiment 1 is followed.

While for the prospective implantation diagnostic in the early to medium secretion phase of the actual cycle tissue samples of the endometrium, the endocervix, and oral mucous membrane or of other select epithelium are examined in order to determine (to decide) the quality of secretory transformation and of the receptivity of the endometrium to be expected (for example, for making a decision on an embryo transfer or insemination in the hormonally stimulated cycle), the retrospective diagnostic of the receptivity of the endometrium (for example, menstrual blood as a non-invasive method) after completion of embryo transfer or after stimulated or non-stimulated cycle represents an important and simple method for making assessments in regard to the secretory transformation of the endometrium of the previous cycle as a diagnostic and/or optionally therapy control and an assessment for the following cycle. Possibly, this method can supplement or replace the customary (clinically employed) invasive method of sample curettage.

### EMBODIMENT 4

For tumor diagnostic, cells are removed from the female patient by a mini catheter from the uterine cavity or removed by a cotton swab from the cervix or by a wooden tongue depressor from the oral mucous membrane. The cells are immediately frozen and stored at −80° C. until further processing. For analysis, the taken-up cells are subjected to a TRIZOL RNA extraction, the cDNA of the endometrial βhCG is amplified specifically in the subsequent RT-PCR process and quantitatively determined.

It can be assumed that the presence of hCG β5, β8, and β6e is an indicator for a tumor disease. The presence of hCG β7, β6 and β3 indicates the opposite: a possible non-trophoblastic tumor disease can be excluded. Of particular importance is the fact that by means of the βhCG diagnostic aggressive tumors can be detected so that the diagnosis also provides a therapy indication. It should be noted that hCG β6 and β6e are represented essentially by hCG β7 (see embodiment 1). The examinations are performed advantageously in the endometrium in order to detect carcinoma therein. Tissue samples can also be obtained in analogy to the method of fractioned curettage.

Endometrial tissue or cells of this origin (10-100 mg) are frozen immediately after their removal in liquid nitrogen or at −80° C. For the examination of the three expressed proportions hCG β7, β6 and β6e and hCG β8, β5, β3 and total βhCG, the total RNA is extracted with TRIZOL and approximately 1 μg of the RNA is reverse transcribed for 60 minutes at 42° C. under standard conditions and use of oligo-dT (15) primer.

Use of methods: tissue removal for diagnostic, storage in liquid nitrogen, RNA extraction (23), RT-PCR with fluorescence-marked primer pair, detection of total βhCG expression β5, β8, β3 and β7, β6 and β6e via exon 1, exon 2, and exon 3, nested PCR method with different fluorescence-marked primers for the β7, β6, β6e and possibly β5, β8, β3 proportions, respectively; quantitative evaluation as a quotient of β7, β6, β6e fluorescence proportion to the total hCG proportion β7, β6 plus β5, β8, β3 proportion for the evaluation of the highly built-up secretory endometrial tissue, result 1 for normal tissue and result >0 to 1 below-value or lacking secretorily transformed tissue in the embodiment 4; or the absolute quantitative evaluation of the expressed copy numbers for the gene-specific βhCG amplified materials β7, β6, β6e and total βhCG according to real-time RT-PCR in comparison to βhCG sequence-specific calibrators for non-fluorescence-marked primers and use of standard methods for the evaluation of the normal and the dard methods for the evaluation of the normal and neoplastic tissue as in embodiment 5.

Use of devices and material: tissue in liquid nitrogen, Ultra Turrax tissue homogenization, TRIZOL RNA extraction, RT-PCR on thermocycler, fluorescence measurement of the cDNA amplified material on DNA sequencer ABI 373A or comparable models, software Genescan 672 fragment analysis for evaluation, liquid nitrogen, TRIZOL, cDNA synthesis kit, PCR amplification kit, βhCG primer for total βhCG amplification and nested RT-PCR for β7, β6 and β6e and β5, β8, β3, partially fluorescence-marked.

Description of method for embodiment 4: extraction of total RNA: the fresh tissue material is frozen immediately after removal in liquid nitrogen. The total RNA is extracted by the method of Chomczynski and Sacchi (24), the obtained RNA is quantified spectrophotometrically at 260 nm/280 nm, immediately processed further or stored at −80° C.

Reverse transcription: 1 μg total RNA is transcribed in a reaction mix with the total volume of 5 μl according to standard method: 10 mM Tris-HCl, pH 8.3, 50 mM KCl, 5 mM MgCl₂, 1 mM each dNTP (dATP, dTTP, dCTP, dGTP), 200 ng oligo dT primer pdT15, 12.5 U RNase inhibitor, 2.5 U AMV revertase. Incubation of the reaction mixture for 10 minutes at 25° C. (hybridization of the primer), 30 minutes at 42° C. (reverse transcription) and 5 minutes at 95° C. (denaturization of the revertase and of the RNase inhibitor) as well as cooling to 4° C.

PCR amplification of the total βhCG transcripts: in the same tube, to the cDNA product the PCR mix of 20 μl is added in a total volume of 25 μl for the amplification of the total βhCG transcript: final concentration of 10 mM Tris-HCl at pH 8.3, 50 mM KCl, 1.5 mM MgCl₂, 200 μM dNTP, 5 pmol primer 1, 5 pmol primer 2, and 2.5 U Taq DNA polymerase. The amplification conditions, after a prior 3 minute incubation at 95° C., are as follows: 30 seconds at 95° C., 30 seconds at 60° C., 60 seconds at 72° C. for 35 cycles with final 7 minutes at 72° C. and fast cooling to 4° C.

Nested PCR for βhCG β7, β6 and β5, β8, β3 transcripts: 2 μl of the PCR product diluted 1:10,000 are added to a total volume of 20 μl in a PCR mix with a final volume of 10 mM Tris-HCl, pH 8.3,10 mM KCl, 3 mM MgCl₂, 50 μM dNTP, 0.1 pmol primer 2, 0.1 pmol primer 3, 0.1 pmol primer 4, and 2 U Taq DNA polymerase (Stoffel fragment). The reaction was carried out for five cycles on a thermocycler for 30 seconds at 95° C. and 30 seconds at 65° C., respectively. The nested PCR reaction is performed also with Taq DNA polymerase at standard conditions.

The obtained product contains the two amplification products for βhCG β7, β6, β6e and possibly of hCG β5, β8, β3 with a different fluorescence marker for primer 4 and primer 3, respectively, and both transcripts contain in addition a third common fluorescence marker of the primer 2.

For the analysis on the DNA sequencer (Perkin-Elmer model 373A or a comparable model) 2.5 μl of the product with 2 μl loading buffer and 0.5 μl Genescan size marker and subjected to the electrophoresis at 8% acrylamide, 6 M urea, and TBE buffer for one hour. The results are analyzed with the Genescan 672 software (Perkin-Elmer) by employing the obtained fluorescence values for total βhCG transcripts and the β7, β6, β6e fragments and possibly the β5, β8, β3 fragments.

The transcription index is calculated in accordance with the method disclosed in Bellet et al. (17).

### EMBODIMENT 5

Tumor tissue (50 to 200 mg) is frozen immediately after removal in liquid nitrogen. For the examination of the three expressed proportions hCG β8, β5, β3 and hCG β7, β6 as well as total βhCG, the total RNA is extracted with TRIZOL and approximately 1 μg of the RNA is reverse transcribed for 60 minutes at 42° C. under standard conditions and use of oligo-dt (15) primer.

For quantitative determination of the three above-mentioned βhCG expression proportions, the real-time PCR on a Light Cycler (Roche) or comparable devices of other firms such as Applied Biosystems is used for the amplification of the tumor cDNA. For the synthesis of the RNA standards of the three βhCG the expression proportions β8, β5, β3 and possibly β7, β6 and the total βhCG, the three corresponding calibration fragments are amplified under standard PCR conditions from endometrial, placental and tumor cDNA. For this purpose, again the three aforementioned now unmarked forward hCG primers (primers 1, 3, 4 or others) specific to βhCG type II (β8, β5, β3), βhCG type I (β7, β6) and total βhCG are used with the common reverse βhCG primer (primer 2 or others). The obtained PCR products are cloned in the plasmid vector pGEM-T. By employing the T7 promoters and Sp6 promoters of the pGEMT-T vector, the plasmid serves as a template for the in-vitro formation of RNA in accordance with the manufacturer's protocol. The formed standard RNAs are cleaned and the concentration is measured.

The real-time PCR amplification on the Light Cycler (Roche) or the ABI systems (Applied Biosystems) determines the number of formed gene copies for the two gene-specific βhCG expression groups type II (β8, β5, β3) and type I (β7, β6) as well as total βhCG in the tumor tissue and in the RNA standards, and, by employing the primers 8, 9, 10 against 2, the individual proportions of ββhCG β5, β6 and β7 can be detected and quantified as an absolute value. The PCR reaction is carried out in the 20 μl reaction volume in the final concentrations of 1× PCR buffer of 50 mM Tris-HCl (pH 8.3), 200 μM dNTPs, with 0.5 μM of the specific forward and reverse βhCG primers, respectively, 4 to 5 mM MgCl_{2β, 0.5} U Taq polymerase, SYBR Green I with 1:3,000 of the master solution (Molecular Probes) and 1 μl of the templates (tumor cDNA or standards of known concentration). Other methods of the real-time RT-PCR (TaqMan, FRET, Beacon) are used also.

The invention also claims the real-time measurement as a one-tube RT-PCR or the use of other methods for quantitative detection of the expression of specific gene copies aside from SYBR Green I, for example, the use of gene-specific oligo nucleotides as hybridization samples with different dye marker or fluorescence marker binding (TaqMan, FRET, Beacon).

### EMBODIMENT 6

Tumor tissue is removed for diagnostic and stored in liquid nitrogen. After RNA extraction (23), an RT-PCR with fluorescence-marked primer pair in accordance with embodiment 4 is carried out. The total βhCG expression β5, β8, β3 and β7, β6 via exon 1, exon 2, and exon 3 is detected with a nested PCR with fluorescence-marked primers for the β7, β6 and the β5, β8, β3 proportions and is evaluated as a quotient of β5, β8, β3 proportion to the β7, β6 plus β5, β8, β3 proportion for the evaluation of the neoplastic and tumorous non-trophoblastic tissue as follows: result 0 for normal tissue and result >0 to 1 for neoplastic tissue—in accordance with embodiment 4. An absolute quantitative evaluation of the expressed copy numbers for the gene-specific βhCG amplified materials β5, β8, β3 as well as β7, β6 and total βhCG according to real-time RT-PCR in comparison to hCG sequence-specific calibrators for the evaluation of the normal and neoplastic tissue in embodiment 5 is carried out.

Use of devices and material: tissue in liquid nitrogen, Ultra Turrax tissue homogenization, TRIZOL RNA extraction, RT-PCR on thermocycler, fluorescence measurement of the cDNA amplified material on DNA sequencer ABI 373A, software Genescan 672 fragment analysis for evaluation, liquid nitrogen, TRIZOL, cDNA synthesis kit, PCR amplification kit, βhCG primer for total βhCG amplification, and nested PCR for β5, β8, β3 and β7, β6, partially fluorescence-marked.

### DESCRIPTION OF THE METHOD FOR EMBODIMENT 6

Extraction of total RNA: the tissue material is frozen in liquid nitrogen immediately after removal. The total RNA is extracted by the method of Chomczynski and Sacchi (24), the obtained RNA is quantified spectrophotometrically at 260 nm/280 nm, immediately processed further or stored at −80° C.

Reverse transcription: 1 μg total RNA is transcribed in a reaction mix with the total volume of 5 μl according to standard method: 10 mM Tris-HCl, pH 8.3, 50 mM KCl, 5 mM MgCl₂, 1 mM each of dNTP(dATP, dTTP, dCTP, dGTP), 200 ng oligo dT primer pdT15, 12.5 U RNase inhibitor, 2.5 U AMV revertase (Roche), incubation of the reaction mixture for 10 minutes at 25° C. (hybridization of the primer), 30 minutes at 42° C. (reverse transcription) and 5 minutes at 95° C. (denaturization of the revertase and of the RNase inhibitor) as well as cooling to 4° C.

PCR amplification of the total βhCG transcripts: in the same tube, to the cDNA product the PCR mix of 20 μl is added in the total volume of 25 μl for the amplification of the total βhCG transcript: final concentration of 10 mM Tris-HCl, pH 8.3, 50 mM KCl, 1.5 mM MgCl₂, 200 μM dNTP, 5 pmol each of the two selected primers and 2.5 U Taq DNA polymerase. The amplification conditions, after prior 3 minute incubation at 95° C., are as follows: 30 seconds at 95° C., 30 seconds at 60° C., 60 seconds at 72° C. for 35 cycles with final 7 minutes at 72 ° C. and fast cooling to 4° C.

Nested PCR for βhCG β7, β6 and β5 and β5, β8, β3 transcripts: 2 μl of the PCR product diluted 1:10,000 are added to a total volume of 20 μl in the PCR mix with a final volume of 10 mM Tris-HCl, pH 8.3,10 mM KCl, 3 mM MgCl₂, 50 μM dNTP, 0.1 pmol primer 2, 0.1 pmol primer 3, 0.1 pmol primer 4, and 2 U Taq DNA polymerase (Stoffel fragment). The reaction was carried out for 5 cycles on a thermocycler for 30 seconds at 95° C. and 30 seconds at 65° C., respectively. The nested PCR reaction is performed also with Taq DNA polymerase at standard conditions.

The obtained product contains the two amplification products for βhCG β5, β8, β3 and β7, β6 with a different fluorescence marker for primer 3 and primer 4, respectively, and both transcripts contain in addition a third common fluorescence marker of the primer 2.

For the analysis on the DNA sequencer (Perkin-Elmer model 373A or comparable models) 2.5 μl of the product with 2 μl loading buffer and 0.5 μl Genescan size marker and subjected to the electrophoresis at 8% acrylamide, 6 M urea, and TBE buffer for one hour. The results are analyzed with the Genescan 672 software (Perkin Elmer) by employing the obtained fluorescence values for total βhCG transcripts and the β7, β6 and β5, β8, β3 fragments.

The transcription index is calculated by the method disclosed in Bellet et al. (17) as the quotient of βhCG β7, β6 relative to the sum of βhCG β7, β6, and β5, 8, β3.

The invention presented here provides a series of important advantages. The obtained results gain in reliability because there are several points of action for the indicator. In contrast to the known technical solution that is based solely on the point mutation 117 in exon 3, our solution includes exon 2 and a promoter gene. Our method enables a differentiation in malignant and in benign tumors with the desired results for a therapy proposal. This is based on the finding that the degree of malignancy of a non-trophoblastic tumor is indicated by the presence of hCG β5, β8, β3. Its concentration is measured in the embodiment 4 as a fluorescence value and brought into relation with hCG β5, β8, β3 in that the quotient of hCG β5, β8, β3 relative to the sum of hCG β5, β8, β3 plus hCG β6, β7 is generated.

In the embodiment 5 the presence of hCG β5, β8, β3 is quantified as an absolute value by real-time RT-PCR by the number of copies of its gene expression in comparison to the sequence-specific βhCG standard series as well as hCG β7, β6.

The method according to the invention is preferably performed by means of a test kit that comprises the following components:

| Reaction solutions | ingredients |
|---|---|
| 1. primer 1 | unmarked primer 1 |
| 2. primer 2 | fluorescence-marked primer 2 |
| 3. primer 3 | fluorescence-marked primer 3 |
| 4. primer 4 | fluorescence-marked primer 4 |
| 5. RT reaction mix | RT reaction buffer with dNTPs, oligo-pdT15, |
| | RNase inhibitor for cDNA formation |
| 6. reverse transcriptase | master solution for RT |
| 7. PCR reaction mix | PCR reaction buffer |
| 8. PCR polymerase | Taq DNA polymerase |
| 9. nested PCR reaction | nested PCR reaction buffer |
| mix | |

The mRNA quantification kit for βhGC gene β5, β7 enables the highly sensitive and specific determination of gene expression of the βhCG in normal and tumor tissue for diagnostic and therapy control.

The specific βhCG β5 and βhCG β7 copies amplified by the methods of real-time RT-PCR can be detected across a wide measuring range by means of a set of provided calibration standards of βhCG β5 mRNA and βhCG β7 mRNA, respectively.

### LIST OF ABBREVIATIONS

cDNA complementary DNA
CTP C-terminal peptide
ELISA enzyme linked immunosorbent assay
ET embryo transfer
hCG human chorionic gonadotropin
α-hCG alpha subunit of hCG
β-hCG beta subunit of hCG
thCG trophoblastically expressed form of hCG
HRP horse radish peroxidase (horseradish peroxidase)
βLH β luteinized hormone
PBS phosphate buffer saline (sodium phosphate buffer)
pdT15 primer poly-deoxythymidine composed of 15 monomers 15
Mab monoclonal antibody
MEIA microparticle enzyme immunoassay
M mol/liter
mRNA messenger RNA
PCR polymerase chain reaction
RT-PCR reverse transcriptase PCR

### CITED NON-PATENT LITERATURE

(1) J. C. Pierce, T. F. Parsons, Annu. Rev. Biochem., 50 (1981) 465-495
(2) P. A. Rothman et al. Mol. Reprod. Dev., 33 (1992) 1-6
(3) S. Dirnhofer et al. J. Clin. Endocrinol. Metab., 81 (1996) 4212-4217
(4) Z. M. Lei et al., J. Clin. Endocrinol. Metab., 77 (1993) 863-972
(5) T. Yokotani et al. Int. J. Cancer, 71 (1997) 539-544
(6) P. Berger et al. FEBS Lett., 343 (1994) 229-233
(7) I. Marcilliac et al., Cancer Res., 52 (1992) 3901-3907
(8) H. Alfthan, et al., Clin. Chem., 38 (1992) 1981-1987
(9) V. Lazar et al., Cancer Res., 55 (1995) 3735-3738
(10) P. N. Span et al., J. Endocrinol., 172 (2002) 489-495
(11) M. Lundin, et al., Int. J. Cancer, 95 (2001) 18-22
(12) K. Hotakainen et al., Brit. J. Cancer, 86 (2001) 185-189
(13) D. S. Hoon, et al., Int. J. Cancer, 69 (1996) 369-374
(17) D. Bellet, et al., Cancer Res., 57 (1997) 516-523
(18) P. K. Hotakainen et al., Mol. Cell. Endocrinol., 162 (2000) 79-85
(19) A. K. Miller-Lindholm, et al., 138 (1997) 5459-5465
(20) S. Madersbacher, et al., Cancer Res., 54 (1994) 5096-5100
(21) R. Oyasu, et al., Arch. Pathol. Lab. Med., 119 (1994) 715-717

What is claimed is:

1.-30. (canceled)

31. A method for determining specific conditions or changes in the endometrium or in the epithelium of other organs, the method comprising the steps of:
a) isolating RNA from a blood sample or tissue sample; and
b) quantitatively measuring in said blood sample or said tissue sample the expression or over expression of mRNA of at least one of β7-hCG, β6-hCG, and β6e-hCG.

32. The method according to claim 31, further comprising the steps of:
c) additionally quantitatively measuring total βhCG mRNA expression or mRNA expression of at least one of β5-hCG, β8-hCG, β3-hCG; and
d) bringing into relation measured values of the step c) with measured values of the step b).

33. The method according to claim 32, wherein in at least one of the steps b) and c) quantitative RT-PCR or real-time RT-PCR is used.

34. The method according to claim 33, wherein, based on the cDNA obtained by reverse transcriptase (RT), total β-hCG cDNA is amplified in the first PCR step with at least one first primer pair comprised of a first primer and a second primer, wherein the first primer pair hybridizes with cDNA of β5-hCG, β8-hCG, β3-hCG as well as β7-hCG and β6-hCG and β6e-hCG, and in a subsequent second PCR step the cDNA of at least one of β7-hCG, β6-hCG, and β6e-hCG is specifically amplified with at least one third primer, wherein the third primer specifically hybridizes with cDNA of β7-hCG and β6-hCG and β6e-hCG, but not with cDNA of β5-hCG, β8hCG, and β3-hCG.

35. The method according to claim 34, wherein in the second PCR step additionally the cDNA of at least one of β5-hCG, β8-hCG, and β3-hCG is specifically amplified with at least one fourth primer, wherein the fourth primer hybridizes specifically with the cDNA of β5-hCG, β8-hCG and β3-hCG but not with the cDNA of β7-hCG and β6-hCG and β6e-hCG.

36. The method according to claim 34, wherein the at least one first primer pair are oligonucleotides selected from the group of sequences consisting of SEQ ID NO. 1, SEQ ID NO. 2, SEQ ID NO. 11, and SEQ ID NO. 14, wherein the third primer is an oligonucleotide selected from the group of sequences consisting of SEQ ID NO. 3, SEQ ID NO. 9, SEQ ID NO. 10, SEQ ID NO. 13, and SEQ ID NO. 16.

37. The method according to claim 35, wherein the fourth primer is an oligonucleotide selected from the group of sequences consisting of SEQ ID NO. 4, SEQ ID NO. 8, SEQ ID NO. 12, and SEQ ID NO. 15.

38. The method according to claim 35, wherein at least one of the first, second, third and fourth primers is fluorescence marked.

39. The method according to claim 38, wherein one of the first and second primers of the first primer pair, the third primer and optionally the fourth primer are provided with fluorescence markers that differ from one another with regard to adsorption and/or emission spectra.

40. The method according to claim 31 for prospective or retrospective diagnostic of an endometrial receptivity for implantation of an embryo.

41. The method according to claim 40, wherein the blood sample is taken from peripheral blood and the tissue sample is taken from tissue of endometrium or cervix of a female patient, wherein, based on the determined mRNA expression of at least one of β7-hCG, β6-hCG, and β6e-hCG, conclusions in regard to receptivity of the uterus for an embryo in the actual cycle are drawn.

42. The method according to claim 40, wherein the blood sample is taken from menstrual blood and, based on determined mRNA expression of at least one of β7-hCG, β6-hCG, and β6e-hCG in the past cycle, prognoses of the potential receptivity of the uterus for an embryo in the subsequent cycle are made.

43. The method according to 31 for tumor diagnosis.

44. The method according to claim 43, wherein, for detecting uterine carcinoma, the tissue sample is removed from the endometrium or cervix of a female patient.

45. The method according to claim 43, wherein values of the mRNA expression in the tissue sample are compared to values of the mRNA expression in healthy tissue.

46. The method according to claim 43, wherein a value of promoter expression of at least one of β5-hCG, β8-hCG, and β3-hCG is determined and is divided by the mRNA expression of total βhCG and, based on the resulting quotient, conclusions in regard to a degree of malignancy of the tumor are drawn.

47. A primer sequence selected from the group consisting to SEQ ID NO. 3, SEQ ID NO. 4, SEQ ID NO. 8 to SEQ ID NO. 16.

48. A diagnostic kit for determining specific conditions or changes in the uterus by quantitative RT-PCR comprising:
a) oligo-dT,
b) enzyme reverse transcriptase,
c) at least two primers hybridizing with cDNA of at least one of β7-hCG, β6-hCG, and β6e-hCG, wherein at least one of the two primers does not hybridize with at least one of β5-hCG, β8-hCG, and β3-hCG,
d) a DNA polymerase resistant above 80° C., and
e) reaction buffer.

49. The diagnostic kit according to claim 48, wherein the at least two primers comprise:
a first primer pair comprised of a first primer and a second primer wherein the first primer pair hybridizes with cDNA of β5-hCG, β8-hCG, and β3-hCG as well as β7-hCG, β6-hCG, and β6e-hCG; and
a third primer that hybridizes specifically with cDNA of β7-hCG and β6-hCG and β6e-hCG but not with cDNA of β5-hCG, β8-hCG, β3-hCG.

50. The diagnostic kit according to claim 49, wherein the at least two primers comprise a fourth primer that hybridizes specifically with cDNA of β5-hCG, β8-hCG, and β3-hCG but not with cDNA of β7-hCG and β6-hCG and β6e-hCG.

51. The diagnostic kit according to claim 49, wherein the first primer pair is selected from the group of sequences consisting of SEQ ID NO. 1, SEQ ID NO. 2, SEQ ID NO. 11 and SEQ ID NO. 14, and wherein the third primer is selected from the group of sequences consisting of SEQ ID NO. 3, SEQ ID NO. 9, SEQ ID NO. 10, SEQ ID NO. 13, and SEQ ID NO. 16.

52. The diagnostic kit according to claim 50, wherein the fourth primer is selected from the group of sequences consisting of SEQ ID NO. 4, SEQ ID NO. 8, SEQ ID NO. 12, and SEQ ID NO. 15.

53. The diagnostic kit according to claim 50, wherein at least one of the first, second, third and fourth primers is fluorescence marked.

54. The diagnostic kit according to claim 53 wherein one of the first and second primers of the first primer pair, the third primer and optionally the fourth primer are provided with fluorescence markers that differ from one another with regard to adsorption and/or emission spectra.

55. The diagnostic kit according to claim 48, comprising a defined amount of mRNA or cDNA of at least one of β5-hCG and β7-hCG as a standard.

56. The diagnostic kit according to claim 48 for prospective or retrospective diagnostic of endometrial receptivity for implantation of an embryo.

57. The diagnostic kit according to claim 48 for tumor diagnosis.

58. A variant β6e of the β6 gene or β7 gene having a nucleic acid sequence SEQ ID NO. 7 and/or coding for a protein with the amino acid sequence selected from the group consisting of SEQ ID NO. 17 and SEQ ID NO. 18.

59. A marker for prospective or retrospective diagnostic of endometrial receptivity for implantation of an embryo, wherein the marker has a gene sequence according to claim 58 or SEQ ID NO. 5 or SEQ ID NO. 6.

60. A marker for tumor diagnostic, wherein the marker has a gene sequence according to claim 58 or SEQ ID NO. 5 or SEQ ID NO. 6.

61. A method for prospective or retrospective diagnostic of endometrial receptivity for implantation of an embryo and for tumor diagnostic by of real-time RT-PCR, the method comprising the step of employing gene sequences SEQ ID NO. 1 to SEQ ID NO. 16 with or without fluorescence marker conjugation for measuring quantitatively gene expression of at least one of β5-hCG, β8-hCG, β3-hCG, β7-hCG, β6-hCG, and β6e-hCG.
